(19) United States
(12) Patent Application Publication
(10) Pub. No.: US 2019/0091446 A1
(43) Pub. Date: Mar. 28, 2019

(54) STRAIN RELIEF AND TUBE MANAGEMENT ASSEMBLY FOR AN INFUSION SET

(71) Applicant: Eli Lilly and Company, Indianapolis, IN (US)

(72) Inventors: Daniel Morris HARTMANN, Arlington, MA (US); Lampros C KOURTIS, Cambridge, MA (US); Sean Matthew PSZENNY, Cambridge, MA (US); Monica Rixman SWINNEY, Stoneham, MA (US)

(21) Appl. No.: 16/095,066

(22) PCT Filed: Apr. 20, 2017
(86) PCT No.: PCT/US2017/028619

§ 371 Date: Oct. 19, 2018

Related U.S. Application Data
(60) Provisional application No. 62/326,248, filed on Apr. 22, 2016.

(51) Int. Cl. A61M 25/02; A61M 5/142

(52) U.S. Cl. CPC A61M 25/02; A61M 2025/0266; A61M 2025/0246; A61M 5/14248

(57) ABSTRACT

The present disclosure relates to a tube management device for an infusion set that includes a holding member attachable to the patient and which defines a holding chamber for the tube. The tube may be engaged and disengaged with the device while the lube is operably connected to convey medication. The device may also allow a) the length of flexible tube controlled by the device and b) the position of the final exit of the tube from the holding member relative to the initial entry of the tube into the holding member to both be selectively adjusted. In some embodiments, tire holding chamber is defined by a plurality of channels which may be either non-intersecting or intersecting channels.

### CROSS REFERENCE TO RELATED APPLICATIONS

This application claims priority to U.S. Provisional Patent Application Ser. No. 62/326,248, filed Apr. 22, 2016 and entitled STRAIN RELIEF AND TUBE MANAGEMENT ASSEMBLY FOR AN INFUSION SET, the entire disclosure of which is hereby expressly incorporated by reference herein.

### BACKGROUND

The present disclosure relates to infusion sets for delivering a medication to a patient. Infusion sets typically include infusion line or tubing that leads from a medication delivery device, such as a pump, to a cannula that is inserted into the subcutaneous tissue of the patient. Such infusion sets are often used to inject insulin or other suitable medications.

A typical infusion set includes a connector hub disposed between the medication pump and the cannula. Connector hubs include two mating parts that allow the pump to be connected and disconnected from the cannula without having to remove the cannula from the subcutaneous tissue. A conventional connector hub includes a female part having a septum and a male part having a needle that pierces the septum when the male and female parts are joined together.

In some infusion sets, the connector hub also functions as an insertion hub with cannula being located at the same hub as the septum/needle assembly connection. In other infusion sets, the connection hub is separate from the insertion hub. In an infusion set having separate connection and insertion hubs, a length of tubing will extend from the connection hub housing the septum/needle assembly to the insertion hub housing the cannula. In both types of infusion sets, a length of tubing will extend from the pump to the connection hub.

In both types of infusion sets, the connector hub typically attaches to the skin of the user with an adhesive. Adhesively securing the hub associated with the cannula to the skin of the user helps reduce the movement of the cannula relative to the tissue of the patient and thereby helps prevent inadvertent withdrawal of the cannula. When a separate insertion hub and connection hub are used, the adhesive attachment of the connection hub to the patient can provide strain relief with regard to the tube leading to the insertion hub. Although such infusion set arrangements can be effective, they also have several shortcomings.

One drawback with a system having a combined connection and insertion hub is that there is no second hub disposed between the pump and the combined connection/insertion hub to provide strain relief on the tubing leading to the hub housing the cannula.

For infusion sets having separate connection and insertion hubs, the length of tubing connecting the connection hub to the insertion hub is typically fixed in length. This fixed length of tubing may not be optimal for all patients. For example, it may be desirable to use a shorter length of tubing for small children than for an adult for optimal comfort when securing the hubs to the patient.

Another drawback is that once the connection hub has been affixed to the body, it cannot be repositioned. Thus, the location and angle at which the tube leaves the hub and the length of tubing between the connection hub and the insertion hub are also fixed. The angle at which the tubing protrudes from the connection hub toward the pump may limit the ability of the user to wear the pump in different locations on their body. The fixed length of tubing between the connection hub and insertion hub may also present difficulties. For example, a relatively long length of tubing may be uncomfortable or become snagged on clothing while a relatively short length of tubing may pull on the insertion site and cause discomfort.

Another drawback is that conventional connection hubs and insertion hubs provide the user with limited choice. For example, infusion sets are typically provided with a fixed length of tubing and the user is limited to a selection of a short tube length (typically between 16 inches (40.6 cm) and 18 inches (45.7 cm)), a medium tube length (typically between 23 inches (58.4 cm) and 24 inches (61.0 cm)), long tube length (typically between 31 inches (78.7 cm) and 32 inches (81.3 cm)) and extra long tube length (typically between 42 inches (106.7 cm) and 43 inches (109.2 cm)). Longer lengths are useful for larger individuals and for those patients who want to wear a pump at a convenient location, such as on a waist belt, and infuse at a remote location, such as a forearm. Users are encouraged to rotate infusion sites to prevent scar tissue formation. Thus, a patient may want to use a long length of tubing for one infusion site and a shorter length for the next infusion site.

Infusion sets are typically purchased in boxes of multiple (e.g., ten) sets and multiple, identical boxes are often purchased in a single order to maximize insurance benefits. As a result, a patient is often locked into a particular length of tubing and would find it cost-prohibitive to purchase multiple infusion sets having different length tubing.

A way to minimize such drawbacks and enhance patient convenience is desirable.

### SUMMARY

The present disclosure provides a versatile device for use with infusion sets that provides for strain relief of the tube and management of excess tube length.

In accordance with a first aspect of the present disclosure, a tube management device is provided for an infusion set having a flexible tube for conveying medication between a reservoir and an infusion site on a patient. The device includes a holding member adapted to be attached to the patient, and the holding member defines at least one holding chamber. A non-terminal portion of the flexible tube can enter and be removed from the at least one holding chamber when the flexible tube is operably connected to convey medication between the reservoir and the infusion site, and: a) the length of flexible tube between an initial entry of the tube into the holding member and a final exit of the tube from the holding member; and b) the position of the final exit of the tube from the holding member relative to the initial entry of the tube into the holding member are both selectively adjustable.

The device may also include a support structure which is adapted to be attached to the patient by a fastener, for example, by an adhesive, a flexible strap, or a clip. The support structure is releasably securable to the holding member, and the holding member can be attached and released from the support structure while the holding member is engaged with the flexible tube.

In some embodiments, the at least one holding chamber includes a plurality of channels defined by the holding member, and each of the channels is adapted to receive the flexible tube. Each channel has two opposing and open ends and an elongate opening extending between and interconnecting the opposing open ends whereby a non-terminal portion of the flexible tube can enter the channel through the elongate opening of each of the plurality of channels.

In a device having a plurality of channels, the device may also include a removable cap which covers the elongate openings of the plurality of channels. Such a device may also include a removable elastomeric layer wherein, when the elastomeric layer is engaged with the holding member, it covers the elongate openings. The elastomeric layer may be separate from the removable cap or be a layer of material disposed on the cap.

In devices having a cap, the cap may be removable and attachable such that the flexible tube is repositionable relative to the plurality of channels while the flexible tube is operably connected to convey medication between the reservoir and the infusion site. In other words, it is not necessary to disconnect the tube from the reservoir or infusion site to reposition the tube within the device in such an embodiment.

In a device having a plurality of channels, the plurality of channels may be adapted to grippingly engage the flexible tube to thereby resist relative movement of the flexible tube and the device when the tube is disposed within one of the plurality of channels.

In a device having a plurality of channels, the device may include at least three parallel channels in some embodiments. Such a device may further include a removable elastomeric layer wherein, when the elastomeric layer is engaged with the holding member, it covers the elongate openings. The plurality of channels may also be adapted to grippingly engage the flexible tube to resist relative movement of the flexible tube and the device when the tube is disposed within one of the plurality of channels. The holding member may include an adhesive layer which is adapted to attach the holding member to the patient.

In some devices having a plurality of channels, the plurality of channels includes at least two intersecting channels. In such an embodiment, the device may also include a removable elastomeric layer wherein, when the elastomeric layer is engaged with the holding member, it covers the elongate openings. Such a device may also be configured such that the plurality of channels includes a first plurality of parallel channels and a second plurality of parallel channels, and at least some of the first plurality of parallel channels intersect at least some of the second plurality of parallel channels. The plurality of channels may also be adapted to grippingly engage the flexible tube to thereby resist relative movement of the flexible tube and the device when the tube is disposed within one of the plurality of channels. The holding member may include an adhesive layer or other fastener adapted to attach the holding member to the patient.

It is additionally noted that all of the embodiments described herein having a holding member may also include a support structure which is adapted to be attached to the patient, for example, by an adhesive, a flexible strap, or a clip. In some embodiments, the support structure is releasably securable to the holding member wherein the holding member can be attached and released from the support structure while the holding member is engaged with the flexible tube.

In accordance with another aspect of the disclosure, a tube management device is provided for an infusion set having a flexible tube for conveying medication between a reservoir and an infusion site on a patient. The device includes a thin, resiliently stretchable sheet of material. A plurality of gripping members are attached to the sheet of material at spaced locations and are adapted to engage the tube to prevent relative movement between the gripping member and the tube. Stretching the sheet of material alters the arrangement of the spaced apart gripping members.

In some embodiments, the sheet of material is a resiliently stretchable fabric and the plurality of gripping members are straps secured to the fabric.

### BRIEF DESCRIPTION OF THE DRAWINGS

The features and advantages of the present disclosure will become more apparent to those skilled in the art upon consideration of the following detailed description taken in conjunction with the accompanying drawings, wherein:

FIG. 1 is a schematic view of an insertion set with a tube management device according to an embodiment;

FIG. 2 is a schematic view of an insertion set with a pair of tube management devices according to another embodiment;

FIG. 3 is a perspective view of a tube management device engaged with a tube according to an embodiment;

FIG. 4 is an exploded view of the tube management device of FIG. 3;

FIG. 5 is a perspective view of the tube management device of FIG. 3 with the tube removed;

FIG. 6 is a perspective view of a tube management device similar to that of FIG. 3 and also including a support structure releasably securable to a holding member;

FIG. 7 is a perspective view of the device of FIG. 6 engaged with a tube;

FIG. 8 is a perspective view of a holding member for another embodiment of a tube management device;

FIG. 9 is a perspective view of a tube management device employing the holding member of FIG. 8;

FIG. 10 is a perspective view of the device of FIG. 9 further including a support structure releasably securable to the holding member;

FIG. 11 is a schematic top view of another embodiment of a tube management device;

FIG. 12 is a view of the tube management device of FIG. 11 in a stretched condition;

FIG. 13 is a schematic end view of the tube management device of FIG. 11;

FIG. 14 is a top view of a support structure configured for use with the embodiments of the tube management device disclosed herein;

FIG. 15 is a partial detail view of the support structure of FIG. 14;

FIG. 16 is a bottom view of a holding member releasably securable to the support structure of FIG. 14; and

FIG. 17 is a partial detail view of the holding member of FIG. 16.

Corresponding reference characters indicate corresponding parts throughout the several views. The exemplifications set out herein illustrate exemplary embodiments of the invention, and such exemplifications are not intended to be exhaustive or to be construed as limiting the scope of the invention to the precise forms disclosed.

### DETAILED DESCRIPTION

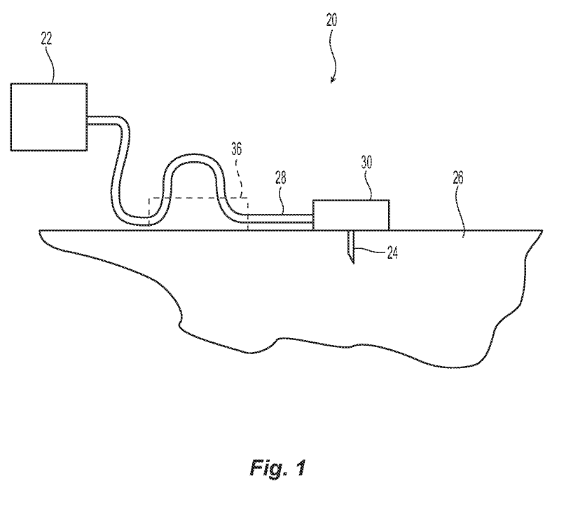

FIG. 1 schematically depicts an infusion set 20 having a cannula 24 to deliver medication from a medication reservoir 22 to a patient 26. Medication reservoir 22 may take the form of a pump operably coupled with a reservoir whereby the pump discharges the medication, such as insulin for example, which flows through flexible tubing 28 to cannula 24 where it is subcutaneously injected into patient 26. Infusion set 20 has a combined connection/insertion hub 30 which is attached to the skin of the patient. A tube management device 36 is attachable to patient 26 and engages flexible tubing 28 at a location between reservoir 22 and hub 30 as schematically depicted in FIG. 1. In one embodiment, flexible tubing 28 is made of a plastic material.

Several embodiments of a tube management device 36 are disclosed herein and discussed in greater detail below. The use of such a tube management device 36 in the fluid path between reservoir 22 and hub 30 allows the user to better control the tubing 28. Further, device 36 is configured to provide strain relief such that if a force is exerted on tube 28 between reservoir 22 and device 36, such as by an accidental snagging of tube 28, the force will be resisted by device 36, and tube 28 will not exert a force on hub 30 which might otherwise dislodge or disconnect cannula 24. For infusion sets having a particularly long length of tubing between reservoir 22 and hub 30, more than one device 36 may be used to control the excess length of tubing.

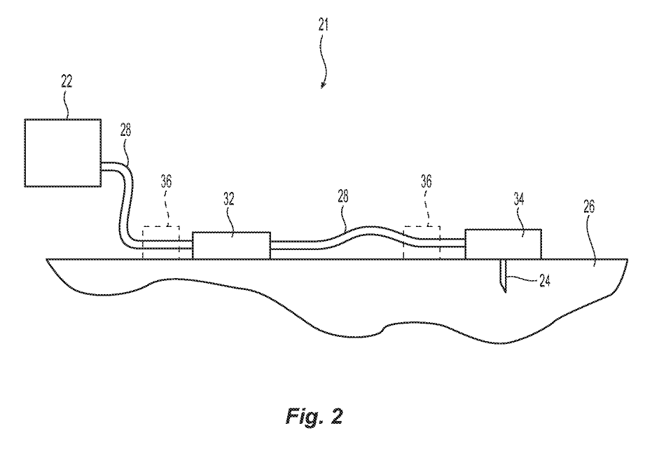

FIG. 2 schematically depicts an infusion set 21 having a connector hub 32 that is physically separate from an insertion hub 34. Connector hub 32 is located in the fluid path between medication reservoir 22 and insertion hub 34 which houses cannula 24. Flexible tubing 28 conveys the medication from reservoir 22 to connection hub 32 and from connection hub 32 to insertion hub 34. Tube management devices 36 may engage tube 28 at locations between reservoir 22 and connection hub 32 and between connection hub 32 and insertion hub 34 as schematically depicted in FIG. 2.

It is noted that infusion sets having separate connection and insertion hubs typically have a relatively short length of tubing between the connection hub 32 and insertion hub 34 that may not require a tube management device. For example, such tube lengths may be between 2 inches (5.1 cm) and 3 inches (7.6 cm). Nevertheless, the use of one or more tube management devices as described herein may be used with the tube at any point in the system where a length of tubing would benefit from the inclusion of such a device.

In an infusion set such as depicted in FIG. 2, tube management devices 36 may be employed both between reservoir 22 and connection hub 32 and between connection hub 32 and insertion hub 34 to meet the needs of the patient.

Both types of infusion sets 20, 21 of FIGS. 1 and 2 have certain advantages. Infusion set 21 with its separate connection hub 32 separates the functionality of connecting and disconnecting the pump from the insertion hub and thereby reduces the likelihood of the act of connecting or disconnecting the pump at the connection hub from disturbing the cannula. More importantly, it also allows the separate connection hub 32 to act as strain relief for the insertion hub 34. However, the tubing between the insertion hub and connection hub is filled with medication and remains connected to the patient even when the rest of the infusion set is removed. The medication contained within this length of tubing (which may be approximately 15 microliters in some infusion sets) may therefore be exposed to different environmental conditions than the remainder of the medication transported by the set. For example, it may be subjected to higher temperatures when the user takes a bath. Moreover, if there is any back-flow from the patient into the infusion set, the medication contained in this length of tubing between the insertion hub and the connection hub may be contaminated and may degrade.

The use of a tube management device 36 between the reservoir and combined hub 30 may provide strain relief for the cannula while eliminating the length of tubing between the insertion hub and connection hub that retains medication when the remainder of the set is disconnected.

By providing a tube management device 36 between the reservoir 22 and combined hub 30, the tube management device 36 is configured to prevent strain from being imparted to the inserted cannula 24. Without such a strain relief device in place, strain may be imparted to the cannula 24 when the user pulls on the infusion tubing 28, which may happen if the user adjusts a waist-belt supporting the pump, snags the tubing on an object, drops the pump, or removes clothing supporting the pump. However, if the tube management device 36 is between the reservoir and combined hub 30 it will absorb such strain and reduce the likelihood of the cannula 24 being dislodged from the patient's body.

The use of a tube management device 36 as described herein eliminates the need for a separate connection hub to provide strain relief and thereby provides strain relief without creating a length of tubing that can lead to the degradation of the medication as described above.

The use of a tube management device 36 also facilitates the use of a relatively long length of tubing between reservoir 22 and either a connection hub 32 or insertion hub 30. The use of a relatively long tube 28 between the connection hub 32 and reservoir 22, or, between the insertion hub 30 and reservoir 22, allows the user greater freedom in choosing the wear location of the reservoir 22 and also greater freedom in selecting the insertion site. The use of such longer tube lengths, however, may present problems when the locations chosen do not require the full length of the tube being used. In such a case, the use of one or more tube management devices 36 is beneficial.

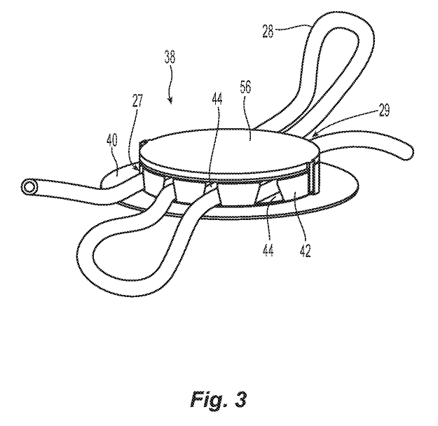

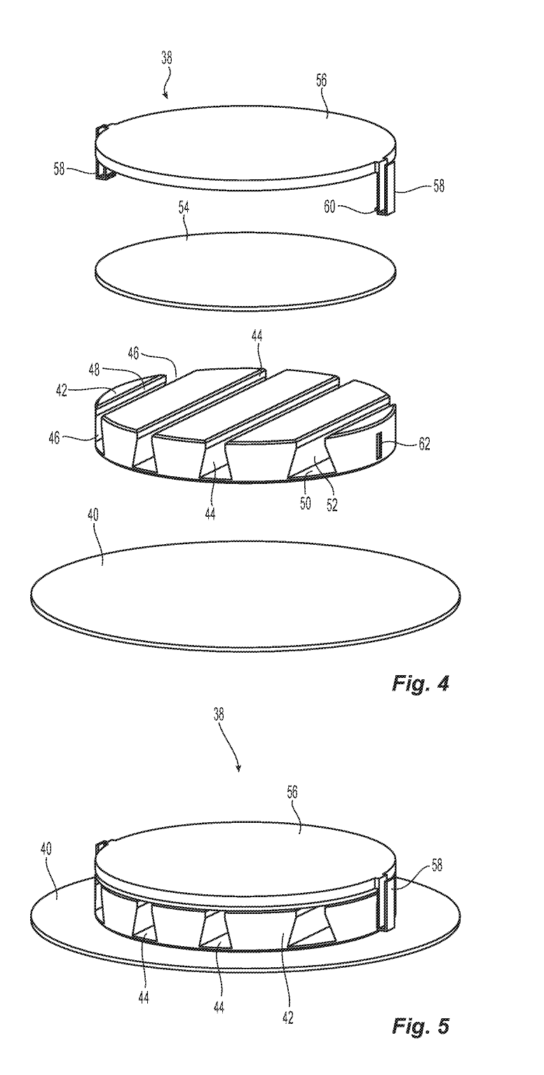

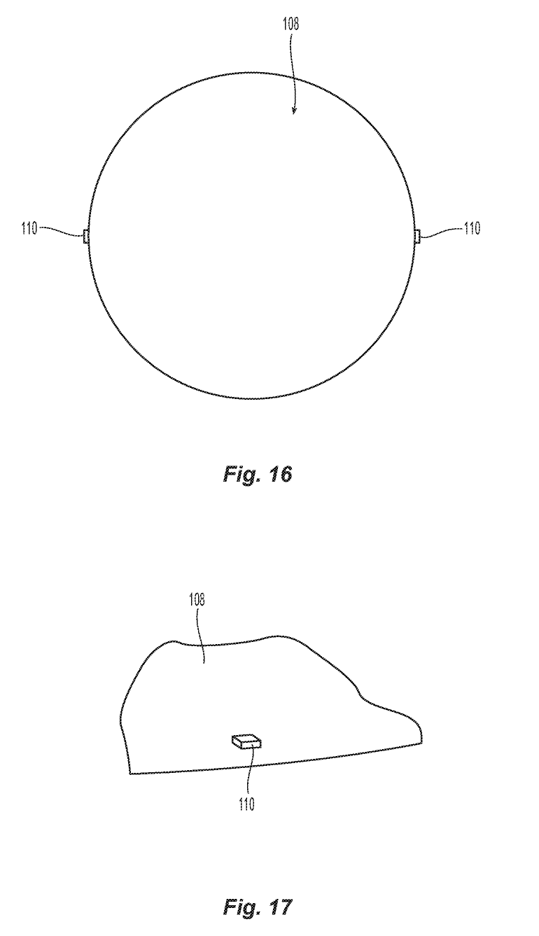

Several embodiments of suitable tube management devices are illustrated in FIGS. 3-17 and are discussed below. These devices can be engaged with tube 28 and disengaged from tube 28 without disconnecting the terminal end of the tube (i.e., without disconnecting reservoir 22, and without disconnecting from the hub 30, 32 or 34), and without having to thread the terminal end of tube 28 through the device. In other words, a non-terminal portion of the flexible tube 28 may be operably engaged with and disengaged from the devices when the flexible tube 28 is operably connected to convey medication between the reservoir and the infusion site.

Because the disclosed devices are engageable with a tube that is already operably connected to the infusion set without disconnecting either end of the tube, the devices may be positioned at any point in the system where there is excess tubing that would be benefit from the use of a management device or a need for strain relief. Multiple devices may also be used for different sections of tubing in the system, or, with a single section of particularly long tubing.

A first embodiment of such a device is depicted in FIGS. 3-5 which illustrates a tube management device 38. Tube management device 38 is suited for use an infusion set having a flexible tube 28 for conveying medication between a reservoir and an infusion site on a patient. Device 38 includes a holding member 42 adapted to be attached to the patient.

The illustrated holding member 42 is secured to the patient by an adhesive layer 40 formed by an adhesive patch. The use of such adhesive patches to secure a medical device to a patient is well known to those having ordinary skill in the art. Although such adhesive patches will typically be the most convenient method to attach device 38, alternative methods could also be employed. For example, holding member 42 may be mounted on a cuff that surrounds and engages a limb of the patient to secure the device. For example, the holding member may be secured to a rectilinear length of fabric, e.g., a stretchable fabric, having cooperating hook and loop patches secured thereto near the ends of the fabric to thereby allow the fabric to be wrapped about the limb of the patient and then secured to itself. Or, in some applications, the device may include a clip for engaging a belt worn about the waist of a patient.

Holding member 42 defines a holding chamber for the tube which takes the form of a plurality of channels 44. Channels 44 are adapted to receive flexible tube 28 with each channel 44 having two opposing and open ends 46 and an elongate opening 48 extending between and interconnecting the opposing open ends 46 whereby a non-terminal portion of flexible tube 28 can enter and be removed from one of the channels 44 through the elongate opening 48 of the channel 44 while flexible tube 28 is operably connected to convey medication between the reservoir 22 and the infusion site 24. In other words, it is not necessary to disconnect one end of tube 28 and insert the disconnected terminal end of the tube through an opening of the channel to engage tube 28 with a channel 44.

Channels 44 are adapted to grippingly engage flexible tube 28 to thereby resist relative movement between flexible tube 28 and device 38 when tube 28 is disposed within one of the plurality of channels 44, For example, channels 44 may be dimensioned to provide an interference fit with tube 28 which slightly compresses tube 28 without interfering with the interior channel of tube 28 when tube 28 is positioned therein. Channels 44 may alternatively be provided with a surface treatment to enhance the frictional engagement of channels 44 with tube 28, or have an arcuate shaped path that biases the tube into engagement with the walls of the channel, or other suitable arrangement to providing a gripping engagement between channel 44 and tube 28.

In the illustrated embodiment, channels 44 have a bottom wall 50 that is wider than elongate opening 48 and sidewalk 52 that slope inwardly as they move upwardly from bottom wall 50 toward elongate opening 48 and engage tube 28 in an interference fit, however, other channel configurations may also be employed.

By providing holding member 42 with at least three parallel channels, device 38 is provided with significant flexibility in how much length of tube 28 is managed by device 38 and the arrangement of tube 28. As can be seen in FIG. 3, by inserting tube 28 into one of the channels 44 and then looping it back to be inserted into another channel 44, the resulting loop may be used to control and manage the excess length of tube 44. By providing at least three channels 44, either one or two loops may be formed. The illustrated example includes four channels 44 allowing up to three such loops to be formed. Alternatively, two channels or any other suitable number of channels may be provided.

The length of flexible tube 28 between its initial entry 27 into holding member 42 and its final exit 29 from holding member 42 may be selectively adjusted both by the altering the channels 44 into which the tube is positioned, and thus altering the number of loops between the entry and exit points 27, 29 of tube 28, and by altering the length of the individual loops.

By having at least three channels 44, the user is provided with a choice as to how many and which channels 44 the tube 28 will be engaged. This also provides the user with a choice as to which openings 46 will define the entry point 27 and exit point 29 of tube 28 from holding member 42 and thereby allowing the position of the final exit 29 of the tube from the holding member relative to the initial entry 27 of the tube into the holding member to be selectively adjustable.

Device 38 also includes a removable cap 56 which closes the elongate openings 48 of the plurality of channels 44. Device 38 also includes a removable elastomeric layer 54. The removable elastomeric layer 54 may be separate from the cap or be a layer of material disposed on the cap and be removable with the cap.

The cap 56 extends over the elongate openings 48 when secured to holding member 42. Elastomeric member 54 covers elongate openings 48 and is held in place by cap 56. Elastomeric member 54 may be formed out of a rubber or other suitable material. Holding member 42 and cap 56 are formed out of an injection moldable thermoplastic material, however, other suitable materials may also be used.

Elastomeric layer 54 reduces relative movement between cap 56 and holding member 42. Depending upon the size of tube 28 and channels 44 and the thickness of elastomeric layer 54, elastomeric layer 54 may also engage a surface of tube 28 through elongate opening 48. When elastomeric layer 54 engages a surface of tube 28, it may grippingly engage the tube to thereby resist relative movement of tube 28 and device 38.

Cap 56 includes a pair of attachment arms 58 located diametrically opposite each other. Arms 58 include a tab 60 that projects inwardly. Tabs 60 engage slots 62 located on holding member 42 to secure both cap 56 to holding member 42. Arms 58 are flexible and may be biased outwardly to disengage tabs 60 from slots 62 to remove cap 56. Various other attachment methods may alternatively be used to removably secure cap 56 to holding member 42. When a separate elastomeric layer 54 is employed, securing cap 56 to holding member 42 also secures the elastomeric layer 54 held between cap 56 and holding member 42.

Cap 56 may be removed and then re-attached to permit tube 28 to be repositioned relative to the plurality of channels 44 while tube 28 is operably connected to convey medication between the reservoir and the infusion site. In other words, it is not necessary to disconnect the tube from the reservoir or infusion site to reposition the tube within the device.

The ability to adjust the length of tubing 28 managed by the device and the entry and exit points of tube 28 without having to disconnect tube 28 provides the user with enhanced flexibility in the positioning of reservoir 22. For example, it may allow the user to reposition the reservoir 22 from a position remote from cannula 24 on the user's body to a closer location and use device 38 to manage the excess tubing that is cause by such a relocation. Similarly, the excess tubing may be released to reposition the reservoir at a more remote location.

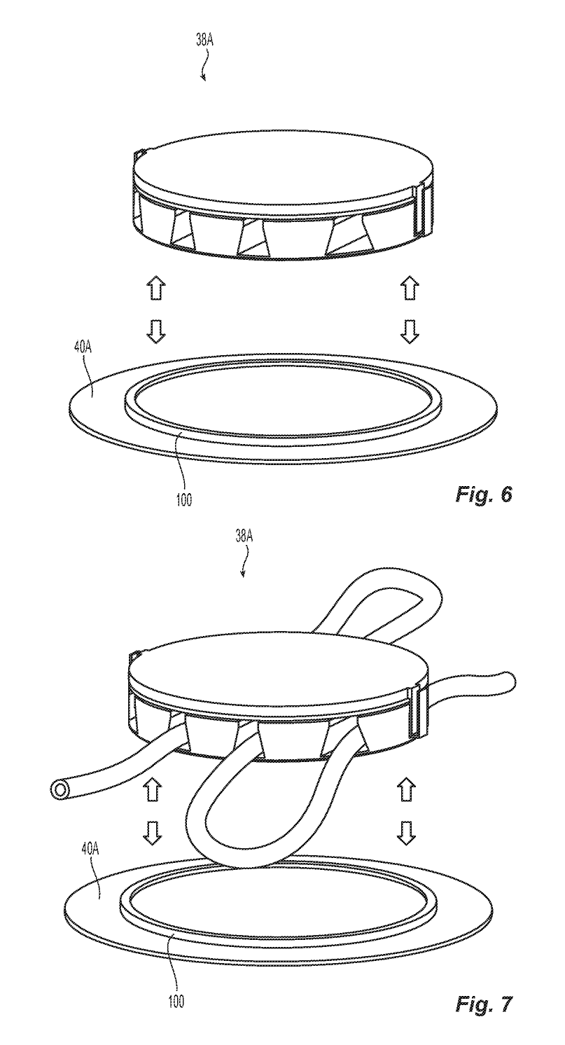

FIGS. 6 and 7 illustrate a modified embodiment of the device depicted in FIGS. 3-5. Device 38A shown in FIGS. 6 and 7 has the same construction as device 38 but further includes a support structure 100. Adhesive patch 40A is adapted to secure support structure 100 to the body of the patient, and support structure 100 is releasably securable to the holding member of device 38A whereby the holding member may be attached and released from support structure 100 while the holding member is engaged with a tube 28. FIGS. 6 and 7 both show the holding member in a detached condition.

The ability to detach the holding member while it is engaged with tube 28 allows the user to disconnect tube 28 from a connection hub and/or insertion hub and then remove the reservoir 22 and associated pump and tube 28 with the holding member still engaged with the tube and without having to disengage tube 28 from the holding member. For example, the user may want to perform such a disengagement of the reservoir and pump when taking a bath. By being able to detach the holding member from the user's body without having to disengage the tube from the holding member, the detachment and subsequent re-attachment process is made simpler and more convenient for the user.

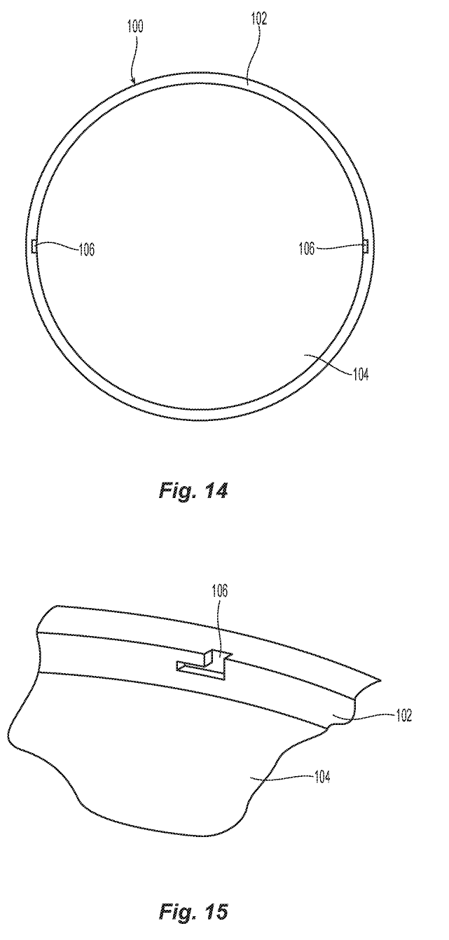

The releasable securement of the holding member with the support structure 100 may be accomplished using a number of different attachment techniques. FIGS. 14-17 illustrate one exemplary manner of releasably securing a holding member 108 to a support structure 100. In this embodiment, support structure 100 includes a ring or collar 102 which extends upwardly from a bottom plate 104. Bottom plate 104 is readily adapted to have an adhesive layer disposed on its surface opposite ring 102. Ring 102 also includes two diametrically opposed L-shaped grooves 106 as best seen in FIG. 15. Holding member 108 includes two diametrically opposed tabs 110 that are inserted into grooves 106. Holding member 108 is then rotated slightly to firmly engage tabs 110 into grooves 106 and releasably secure holding member 108 to support structure 100. To remove, holding member 108 is rotated to a position that allows the withdrawal of tabs 110 from grooves 106.

A variety of other attachment methods may also be employed. For example, a bayonette type attachment, cooperating helical threads, locking tabs similar to tabs 60 and 62, magnetic closures, and/or the use of cooperating hook and loop patches could all be employed. The person having ordinary skill in the art will realize that this listing is exemplary and not exhaustive.

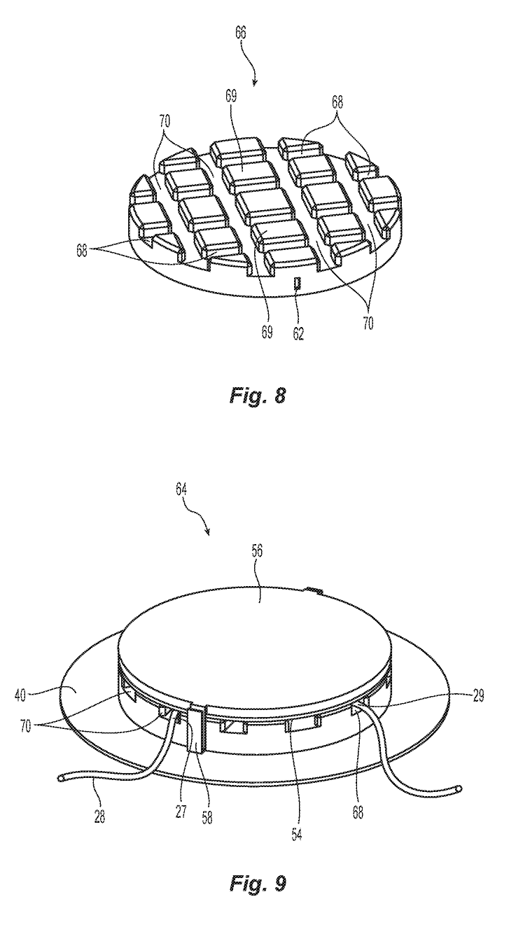

Another alternative embodiment of a tube management device 64 is depicted in FIGS. 8 and 9. FIG. 8 illustrates a holding member 66 of tube management device 64. Device 64 is generally similar to device 38 and uses a removable cap 56 to secure an elastomeric layer 54 to cover channels located in the holding member. Cap 56 includes arms 58 with tabs 60 that engage slots 62 in the holding member 66. An adhesive patch 40 is used to secure holding member 66 to the patient, although alternative fasteners may be used.

Device 64 differs in that holding member 66 includes a plurality of channels 68, 70 wherein some of the channels intersect. Various different arrangements of intersecting channels may be employed. In the illustrated embodiment, holding member 66 includes a first plurality of parallel channels 68 and a second plurality of parallel channels 70 wherein channels 68 intersect channels 70. The use of such intersecting channels provides greater versatility in selecting the angular separation of where tube 28 enters and leaves device 64. Channels 68, 70 are illustratively perpendicular to each other but may be arranged at other suitable angles.

Similar to channels 44 of FIGS. 3-7, channels 68, 70 have two opposing and open ends that are interconnected by an elongate opening that extends therebetween to thereby allow a non-terminal portion of the flexible tube 28 to enter and be removed from the channel when the flexible tube is operably connected to convey medication between the reservoir and the infusion site.

In the illustrated embodiment, the shape of channels 68, 70 also differs from the shape of channels 44, although channels 68, 70 may alternatively have a similar shape as channels 44. Illustratively, instead of having inwardly sloping sidewalls, channels 68, 70 have sidewalls that are oriented perpendicular to the bottom wall of the channel such that the bottom wall and elongate opening along the top of the channel have the same width. The spacing of the sidewalls may be such that the sidewall grip tube 28 in an interference fit when tube 28 is positioned therein. Alternatively, the depth of the channels may be such that attachment of cap 56 slightly compresses the tube 28 and grips the tube 28 between the bottom of the channel and cap 56.

In this regard, it is noted that an elastomeric layer, such as layer 54, will generally provide greater frictional resistance to relative movement of tube 28 than a thermoplastic material. Thus, engagement of layer 54 with tube 28 will not necessarily require compression of tube 28 to grippingly engage tube 28. Furthermore, it is noted that the channel surfaces may have a surface layer that facilitates the gripping engagement of tube 28 without compression of tube 28.

It is also noted that positioning tube 28 in channels 68, 70 such that it bends about one of the raised portions 69 between the channels to move from a channel 68 to a channel 70 will also serve the purpose of gripping tube 28 so that it resists relative movement between the holding member and tube. In this regard, it is further noted that while the illustrated channels 44, 68, 70 are all linear, such channels may alternatively be formed with an arcuate portion, such as a channel defining a serpentine path, whereby inserting the tube in any one such channel will generally be sufficient to prevent relative movement between the tube and the holding member.

The gripping of tube 28 by the device in a manner to resists relative movement between tube 28 and the device not only facilitates the ability of the device to manage excess tube length but also facilitates its functioning as a strain relief device.

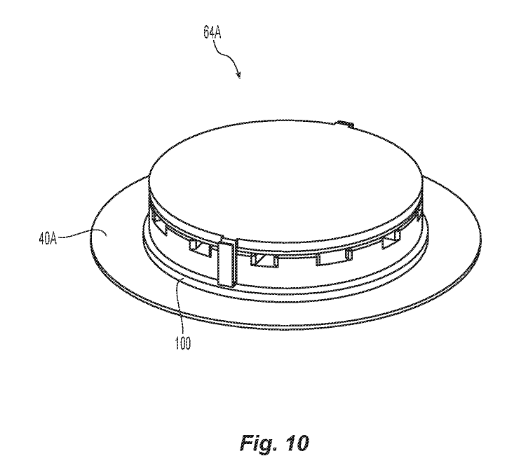

FIG. 10 illustrates a modified embodiment of the device of FIGS. 8 and 9. Device 64A illustrated in FIG. 10 has a same design to that of device 64 but further includes a support structure 100 to which the holding member is releasably attachable. In this embodiment, support structure 100 is secured to the patient with an adhesive patch 40A. Device 64A is shown with the holding member in its attached condition in FIG. 10. Similar to device 38A, device 64A may have its holding member attached and detached from support structure 100 while the holding member is still engaged with a tube 28. Exemplary attachment techniques of a holding member to a support structure are described herein.

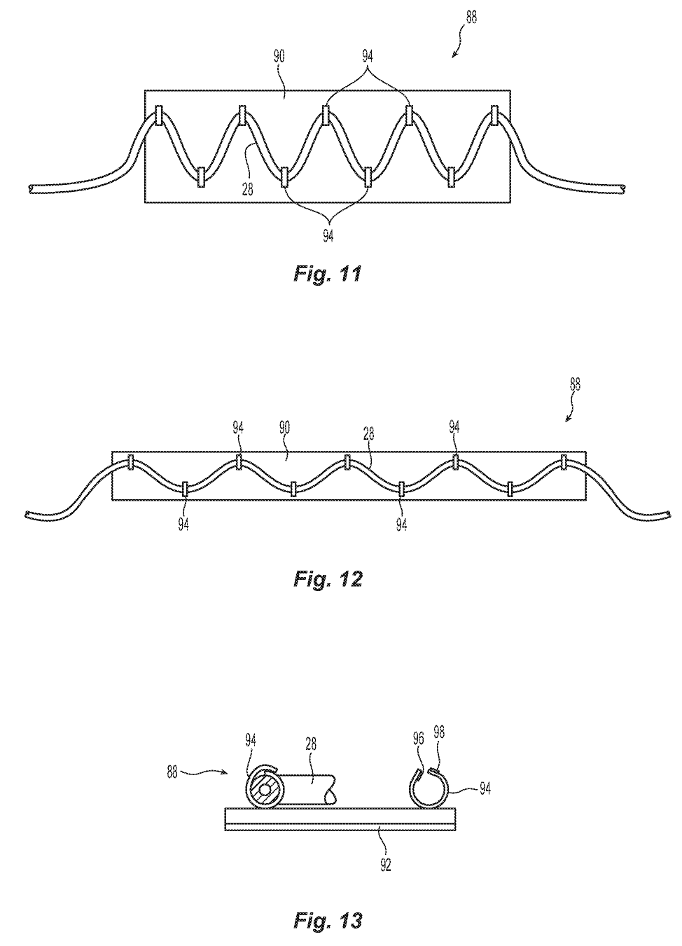

Another embodiment of a tube management device is depicted in FIGS. 11-13. Tube management device 88 is adapted for use with an infusion set having a flexible tube for conveying medication between a reservoir and an infusion site on a patient.

Device 88 includes a thin, resiliently stretchable sheet of material 90. An adhesive layer 92 is disposed on the sheet of material and is used to attach the sheet of material to the patient. A plurality of gripping members 94 are attached to the sheet of material 90 at spaced locations and are adapted to engage tube 24 to prevent relative movement between the gripping member 94 and the tube 28.

FIG. 11 shows the sheet of material 90 in an unstretched condition while FIG. 12 shows sheet of material 90 in a stretched condition. As can be understood with reference to FIGS. 11 and 12, by attaching tube 28 to a plurality of spaced apart gripping members 94 with tube 28 in a serpentine configuration while sheet 90 is in its un-stretched condition, subsequent stretching of sheet 90 allows tube 28 to take a more elongate configuration. As can be seen in FIGS. 11, 12, the arrangement of spaced apart gripping members 94 is altered as the sheet 90 is stretched. This allows the user to change and move positions and have sheet 90 and attached tube 28 adapt to the user's changing position.

It is noted that in FIGS. 11, 12, each of the gripping members 94 is engaged with tube 28. It is also possible, however, to attach tube 28 to only some of the gripping members 94 and thereby control a shorter length of tube 28 and/or alter the locations where tube 28 is initially secured on sheet 90 and the last attachment location on sheet 90.

In the illustrated embodiment, sheet of material 90 is a resiliently stretchable fabric formed using elastic fibers such as fibers made out of a polyester-polyurethane copolymer. Alternative materials may also be used to form sheet 90.

The exemplary gripping members 94 are fabric straps secured at their midpoint to sheet 90 and have cooperating patches of hook fasteners 96 and loop fasteners 98 located at their distal ends as most easily seen in FIG. 13. Hook and loop fasteners 96, 98 may be secured together with tube 28 positioned within the opposite arms of the strap to thereby have the strap tightly grasp and secure tube 28. Alternative methods of securing tube 28 may also be employed. For example, the ends of flexible straps may be tied together to secure tube 28, or rigid body clamping members may be attached to sheet 90 to form the gripping members 94.

As may be seen with reference to FIG. 13, gripping members 94 are located on one major surface of sheet 90 while the fastener, such as an adhesive 92, is located on the other major surface. Advantageously, adhesive 92 is applied in discrete and spaced apart areas to facilitate stretching of sheet 90. For example, adhesive 92 may be applied in small areas corresponding to the locations of gripping members 94. An elastomeric adhesive or other suitable fastener that allows for stretching of material 90 may alternatively be employed.

Alternatively, adhesive 92 is omitted from device 88. Such a device functions solely as a tube management device that is not secured relative to the patient's body and provides management of excess tube length without providing strain relief. Or, device 99 may be secured relative to the patient using some method other than an adhesive, such as a cuff secured about the limb of a patient or by being clipped to a belt or otherwise secured to an article of clothing being worn by the patient.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A tube management device for an infusion set having a flexible tube for conveying medication between a reservoir and an infusion site on a patient, the device comprising:
a holding member adapted to attach to the patient wherein the holding member defines at least one holding chamber adapted to receive a non-terminal portion of the flexible tube when the flexible tube is operably connected to convey medication between the reservoir and the infusion site, the non-terminal portion of the flexible tube being removeable from the at least one holding chamber when the flexible tube is operably connected to convey medication between the reservoir and the infusion site, and wherein: a) the length of flexible tube between an initial entry of the tube into the holding member and a final exit of the tube from the holding member, and b) the position of the final exit of the tube from the holding member relative to the initial entry of the tube into the holding member are both selectively adjustable.

2. The device of claim 1, further comprising a support structure adapted to be attached to the patient, and wherein the support structure is releasably securable to the holding member wherein the holding member is attachable and releasable from the support structure while the holding member is engaged with the flexible tube.

3. The device of claim 1, wherein the at least one holding chamber comprises a plurality of channels defined by the holding member wherein each of the channels is adapted to receive the flexible tube, each channel having two opposing and open ends and an elongate opening extending between and interconnecting the opposing open ends, wherein a non-terminal portion of the flexible tube may enter the channel through the elongate opening of each of the plurality of channels.

4. The device of claim 3, further comprising a removable cap which covers the elongate openings of the plurality of channels.

5. The device of claim 4, further comprising a removable elastomeric layer positioned between the removable cap and the holding member to cover the elongate openings.

6. The device of claim 4, wherein the cap is removable and attachable, and wherein the flexible tube is repositionable relative to the plurality of channels when the flexible tube is operably connected to convey medication between the reservoir and the infusion site.

7. The device of claim 3, wherein the plurality of channels are adapted to grippingly engage the flexible tube to resist relative movement of the flexible tube and the device when the tube is disposed within at least one of the plurality of channels.

8. The device of claim 3, wherein the plurality of channels comprises at least three parallel channels.

9. The device of claim 7, further comprising a removable elastomeric layer wherein, when the elastomeric layer is engaged with the holding member, it covers the elongate openings.

10. The device of claim 3, wherein the holding member includes an adhesive layer is adapted to attach the holding member to the patient.

11. The device of claim 3, wherein the plurality of channels comprises at least two intersecting channels.

12. The device of claim 11, further comprising a removable elastomeric layer wherein, when the elastomeric layer is engaged with the holding member, it covers the elongate openings.

13. The device of claim 11, wherein the plurality of channels includes a first plurality of parallel channels and a second plurality of parallel channels wherein at least some of the first plurality of parallel channels intersect at least some of the second plurality of parallel channels.

14. The device of claim 11, wherein the plurality of channels are adapted to grippingly engage the flexible tube to resist relative movement of the flexible tube and the device when the tube is disposed within one of the plurality of channels and the holding member includes an adhesive layer which is adapted to attach the holding member to the patient.

15. A tube management device for an infusion set having a flexible tube for conveying medication between a reservoir and an infusion site on a patient, the device comprising:
a thin, resiliently stretchable sheet of material; and a plurality of gripping members attached to the sheet of material at spaced locations and adapted to engage the tube to resist relative movement between the gripping member and the tube and wherein a stretching of the sheet of material alters the arrangement of the spaced apart gripping members.

16. The device of claim 15, wherein the sheet of material is a resiliently stretchable fabric and the plurality of gripping members are straps secured to the fabric.
